(19) United States
(12) Patent Application Publication
(10) Pub. No.: US 2021/0315197 A1
(43) Pub. Date: Oct. 14, 2021

(54) PRESERVATION OF PHAGE CONCENTRATION IN CLINICAL SAMPLES

(71) Applicant: Adaptive Phage Therapeutics, Inc., Gaithersburg, MD (US)

(72) Inventor: Carl Merril, Bethesda, MD (US)

(73) Assignee: Adaptive Phage Therapeutics, Inc., Gaithersburg, MD (US)

(21) Appl. No.: 17/129,527

(22) PCT Filed: Jun. 26, 2019
(86) PCT No.: PCT/US2019/039351

§ 371 Date: Dec. 21, 2020

Related U.S. Application Data
(60) Provisional application No. 62/690,417, filed on Jun. 27, 2018.

(51) Int. Cl. A01N 1/02

(52) U.S. Cl. CPC A01N 1/0221; A01N 1/0284

(57) ABSTRACT

A simple method and kit for preserving clinical specimens and other biological samples, particularly for the purpose of preserving the concentration of bacteriophage and/or bacteria present in a sample, is described. The method involves rapidly lowering the temperature of the sample (e.g. freezing) to a temperature of −50° C. or lower (especially about −78° C.) in the presence of a suitable cryoprotectant (e.g. 20% glycerol). In one application, the method is used with clinical samples (e.g. urine) taken from a patient undergoing phage therapy for a bacterial infection, wherein the method permits the samples to be transported and/or stored following collection such that the viability of any phage and/or bacteria present is maintained while also inhibiting potential interactions between the phage and bacteria. After thawing of the samples, the samples may be analyzed for phage and/or bacterial concentration to provide information on the effectiveness of the phage therapy.

### BACKGROUND OF THE INVENTION

### Field of the Invention

The invention relates to a simple method of preserving clinical specimens and other biological samples, particularly for the purpose of preserving the concentration of bacteriophage and/or bacteria present in a sample.

### Discussion of the Related Art

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Multiple drug resistant (MDR) bacteria are emerging at an alarming rate. Currently, it is estimated that at least 2 million infections are caused by MDR organisms every year in the United States leading to approximately 23,000 deaths. Moreover, it is believed that genetic engineering and synthetic biology may also lead to the generation of additional highly virulent microorganisms.

For example, Staphylococcus aureus are gram positive bacteria that can cause skin and soft tissue infections (SSTI), pneumonia, necrotizing fasciitis, and blood stream infections (i.e. bacteremias). Methicillin-resistant S. aureus (“MRSA”) is an MDR organism of great concern in the clinical setting as MRSA is responsible for over 80,000 invasive infections, close to 12,000 related deaths, and is the primary cause of hospital acquired infections. Additionally, the World Health Organization (WHO) has identified MRSA as an organism of international concern.

In view of the potential threat of rapidly occurring and spreading virulent microorganisms and antimicrobial resistance, alternative clinical treatments against bacterial infection are being developed. One such potential treatment for MDR infections involves the use of phage. Bacteriophages (“phages”) are a diverse set of viruses that replicate within and can kill specific bacterial hosts. The possibility of harnessing phages as an antibacterial agent was investigated following their initial isolation early in the 20th century, and they have been used clinically as antibacterial agents in some countries with some success. Notwithstanding this, phage therapy was largely abandoned in the United States after the discovery of penicillin, and only recently has interest in phage therapeutics been renewed.

The successful therapeutic use of phage depends on the ability to administer a phage strain that can kill or inhibit the growth of a bacterial isolate associated with an infection. In addition, given the mutation rate of bacteria and the narrow host range associated with phage strains, a phage strain that is initially effective as an antibacterial agent can quickly become ineffective during clinical treatment as the initial target bacterial host either mutates or is eliminated and is naturally replaced by one or more emergent bacterial strains that are resistant to the initial phage employed as an antibacterial agent.

Accordingly, there is a need to monitor the efficacy of phage therapy by regularly testing a treated patient for changes in phage and/or bacterial concentration in relevant samples. However, any delay between the taking of a patient sample and analysis of that sample such as, for example, the delay caused by the need to transport the sample from the place that it was taken from the patient (e.g. at a hospital) to a suitable external testing laboratory, can lead to significant changes in the phage and bacterial content due to, for example, continued interaction between the phage and bacteria and/or their further replication and population expansion, unless steps are taken to “preserve” the phage and bacteria as they were when the sample was initially obtained. Thus, in order to avoid inaccurate and/or misleading results being obtained from a patient sample, in some previous methodologies, samples containing phage have been preserved by freezing at low temperatures with or without the presence of glycerol (e.g. −5° C.-22° C.; Steele P R M et al., J Hyg 67:679-690, 1969, and Nyiendo J et al., Appl Microbiol 27(1):72-77, 1974, respectively) and/or by adding to the samples one or more antibody selected to neutralize a particular phage type (i.e. strain) and thereby inhibit (or “stop”) interactions between the phage and any bacteria present in the sample. However, these methodologies may produce inconsistent results or fail to fully stop phage replication while the sample is being transported or stored. In addition, the use of neutralizing antibodies introduces complexity and cost to the task (nb. multiple specific neutralizing antibodies need to be employed where multiple different phage strains are involved). Also, once neutralizing antibodies are used, then it is no longer possible to assay for infectious (i.e. “viable”) phage as they can no longer bind or interact with their bacterial hosts (i.e. due to the presence and activities of the neutralizing antibodies).

Thus, there is a need to develop novel and simple methods of preserving clinical specimens comprising phage (e.g. by substantially preserving the concentration of viable phage and/or bacteria as they were when a sample such as a urine sample was initially obtained), desirably without the use of phage neutralizing antibodies, to allow for, for example, monitoring of the efficacy of a phage therapy.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The invention relates to a simple method of preserving clinical specimens and other biological samples, particularly for the purpose of preserving the concentration of bacteriophage that may be present or otherwise for preserving the concentration of bacteria in the presence of bacteriophage.

More particularly, the invention relates to a method of preserving a biological sample comprising, or suspected of comprising, one or more phage, the method comprising rapidly lowering the temperature of the sample (e.g. freezing) to a temperature of −50° C. or lower in the presence of a suitable cryoprotectant.

The biological sample may be selected from clinical specimens and other biological samples such as veterinary samples.

In a particular application of the invention, the method is used with clinical specimens such as blood, serum, plasma, or urine (especially those comprising a mixture of one or more phage and their host bacteria) immediately after collection (e.g. at the patient's bedside) for later analysis.

Similarly, the method may be used with veterinary samples such as blood, serum, plasma, or urine taken from an animal subject (e.g. a livestock animal, companion animal such as a dog or cat, or exotic animal such as a lion or elephant).

Since the method does not require the use of phage neutralizing antibodies, the method is not constrained to any particular phage and, indeed, may be used to preserve one or more phage types (e.g. different phage strains) present in a biological sample. This provides a significant benefit for the analysis of, for example, clinical specimens taken from a patient that may be undergoing phage therapy involving multiple different phage strains (e.g. 2 to 5) as may be sourced from a phage collection or “library”. This would, most likely, not be feasible using phage neutralizing antibodies since the specificity of antibody binding would necessitate the costly use of multiple different phage neutralizing antibodies. Moreover, by avoiding the use of phage neutralizing antibodies, the phage remain viable (i.e. they remain infectious), which means that the preserved biological sample can be readily analyzed for phage concentration (and bacterial concentration if desired), whereas phage bound to neutralizing antibodies are no longer infectious and cannot readily be used to measure phage concentrations. Accordingly, the use of a method to “preserve” phage containing samples with phage neutralizing antibodies may limit analysis of the biological sample to the measurement of bacterial cell concentrations.

The method of the invention involves rapidly lowering the temperature of the biological sample to a temperature of −50° C. or lower, preferably to a temperature of about −78° C. or lower. The temperature of dry ice (frozen carbon dioxide), which is typically readily available in a hospital or laboratory setting for instance, has a temperature of −78.5° C. Accordingly, one of skill in the art will readily appreciate that in some embodiments, the method may involve placing the biological sample into a container (e.g. a polystyrene foam cooler box) of dry ice. In the case of clinical specimens, this can be readily done at the patient's bedside immediately after the sample has been taken; thereby rapidly lowering the temperature of the sample to a temperature of about −78° C.

At a time preferably prior to the step of rapidly lowering the temperature of the sample, a suitable cryoprotectant is added. The cryoprotectant may function to prevent phage and bacteria present in the sample from freezing damage (i.e. damage caused by the formation of ice on or within their structures). More particularly, the cryoprotectant should prevent any bacteria that may be present from being killed by the freezing, otherwise the subsequent analysis of the bacterial concentration sample might lead to an incorrect conclusion that the lack of viable bacteria was due to phage activity. The cryoprotectant may also inhibit any damage to the phage that may affect phage viability.

In some preferred embodiments, the cryoprotectant is glycerol. The glycerol may be added in an amount of, for example, 20% (v/v) (i.e. 20% glycerol).

Once the temperature of the sample has been lowered to a temperature of −50° C. or lower in the presence of a cryoprotectant, it is maintained substantially at such a temperature(s) until required for analysis. As such, during any transport (e.g. shipping) of the sample and any period(s) of storage (e.g. storage before, during and/or after transport), the sample is maintained substantially at a temperature(s) of −50° C. or lower in the presence of a cryoprotectant. Where the sample is placed in a polystyrene foam container with dry ice and then sealed, the temperature of the sample should remain at about −78° C. for up to days and, even weeks, without any additional refrigeration.

Analysis of the sample may involve, for example, one or more of the standard laboratory techniques known to one of skill in the art for assaying for phage and/or bacteria. For phage, this may simply involve preparing serial dilutions of the sample in a suitable media or solution and plating each out on a culture plate provided with a bacterial “lawn”. Following incubation of the plates under suitable conditions, phage plaques can then be scored. Similarly, for bacteria, analysis may involve, for example, preparing serial dilutions of the sample in a suitable media or solution and plating each out on a culture plate. Following incubation of the plates under suitable conditions, bacterial colonies can then be scored.

Where the method of the invention is being applied to clinical specimens taken from a patient undergoing phage therapy, the results of the analysis of the sample can provide, for example, valuable information to the physician on the effectiveness of the phage therapy on the patient's bacterial infection. Subsequently, the physician may to choose to maintain the current phage therapy or otherwise make one or more changes (including changing one or more of the phage strains and/or adding one or more additional phage strains).

The invention further relates to a kit comprising at least a container adapted to receive a biological sample, such as blood, serum, plasma, or urine, wherein said container may be pre-loaded with a suitable cryoprotectant, and wherein the kit optionally includes instructions for use of the kit in the method of invention for preserving a biological sample.

### BRIEF DESCRIPTION OF THE FIGURES

The objects and features of the invention can be better understood with reference to the following detailed description and accompanying drawings.

FIG. 1 provides graphical results obtained from an experiment to assess the interaction between phage and bacterial host cells in samples stored at varying temperatures. Specimens were placed in 0° C. (ice) and −78° C. (dry ice) and at room temperature (RT) and sampled in quintuplicate at 1 hour and at 24 hours. The initial phage titer was 1.8×10⁶ PFU/mL. Error bars represent the standard deviations.

FIG. 2 provides graphical results obtained from a further experiment to assess the interaction between phage and bacterial host cells in samples stored at varying temperatures. Specimens were placed in 0° C. (ice) and −78° C. (dry ice) and at room temperature (RT) and sampled in quintuplicate at 1 hour and at 24 hours. The initial bacterial concentration was 8.8×10⁴ CFU/mL. Error bars represent standard deviations.

FIG. 3 provides graphical results showing phage titers in urine containing 20% glycerol measured from: the initial sample, 1 hour in dry ice (−78° C.) and after 24 hours in dry ice (−78° C.) in acidic, neutral and basic urine samples. Phage titers are expressed as PFUs/mL for each sample. Error bars represent the standard deviations.

FIG. 4 graphically shows the results obtained from an experiment to assess the preservation of bacterial concentrations in urine containing 20% glycerol measured from: the initial sample, 1 hour in dry ice (−78° C.) and after 24 hours in dry ice (−78° C.) in acidic, neutral and basic urine samples. Bacterial concentrations are expressed as CFUs/mL for each sample. Error bars represent the standard deviations.

### DETAILED DESCRIPTION

The following definitions are provided for specific terms which are used in the following written description.

### Definitions

As used in the specification and claims, the singular form “a”, “an” and “the” include plural references unless the context clearly dictates otherwise. Also, as understood by one of skill in the art, the term “phage” can be used to refer to a single phage or more than one phage.

The present invention can “comprise” (open ended) or “consist essentially of” the components of the present invention. As used herein, “comprising” means the elements recited, or their equivalent in structure or function, plus any other element or elements which are not recited. The terms “having” and “including” are also to be construed as open ended unless the context suggests otherwise.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term “about” means within an acceptable error range for the particular value, such as +1-20%, preferably +1-10% and more preferably ±1-5%. In even further embodiments, “about” should be understood to mean +/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by one of skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition is described as containing characteristics A, B, and/or C, the composition can contain A feature alone; B alone; C alone; A and B in combination; A and C in combination; B and C in combination; or A, B, and C in combination.

As used herein, the term “determining” encompasses a wide variety of actions. For example, “determining” may include calculating, computing, processing, deriving, investigating, looking up (e.g. looking up in a table, a database or another data structure), ascertaining and the like. Also, “determining” may include receiving (e.g. receiving information), accessing (e.g. accessing data in a memory) and the like. Also, “determining” may include resolving, selecting, choosing, establishing and the like.

As used herein, “phage therapy” refers to any therapy to treat a bacterial infection or bacterial-caused disease, which may involve the administration to a subject requiring treatment (e.g. a patient) of one or more therapeutic composition that can be used to infect, kill or inhibit the growth of a bacterium, which comprises one or more viable phage as an antibacterial agent (e.g. a composition comprising one phage strain or a phage “cocktail”) and which may further comprise, or otherwise be administered in combination with a further therapeutic composition comprising, one or more antibiotics, one or more bactericides, and/or one or more other therapeutic molecules such as small molecules or biologics that have bactericidal activity. Where more than one therapeutic composition is involved in the phage therapy then the compositions may have a different host range (e.g. one may have a broad host range and one may have a narrow host range, and/or one or more of the compositions may act synergistically with one another). Further, as understood by one of skill in the art, the therapeutic composition(s) used in a phage therapy will also typically comprise a range of inactive ingredients selected from a variety of conventional pharmaceutically acceptable excipients, carriers, buffers, and/or diluents. The term “pharmaceutically acceptable” is used to refer to a non-toxic material that is compatible with a biological system such as a cell, cell culture, tissue, or organism. Examples of pharmaceutically acceptable excipients, carriers, buffers, and/or diluents are familiar to one of skill in the art and can be found, e.g. in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water and saline.

The invention particularly relates to a method of preserving a biological sample comprising, or suspected of comprising, one or more phage, the method comprising rapidly lowering the temperature of the sample (e.g. freezing) to a temperature of −50° C. or lower in the presence of a suitable cryoprotectant, particularly for the purpose of preserving the concentration of bacteriophage that may be present or otherwise for preserving the concentration of bacteria in the presence of bacteriophage.

The biological sample may be selected from clinical specimens and other biological samples such as veterinary samples, agricultural samples and environmental samples.

In a particular application of the invention, the method is used with clinical specimens such as blood, serum, plasma, or urine (especially those comprising a mixture of one or more phage and their host bacteria) immediately after collection (e.g. at the patient's bedside) for later analysis. The patient may be undergoing phage therapy and the clinical specimens taken for the purpose of analyzing the phage and/or bacterial concentration to provide valuable information to the physician on the effectiveness of the phage therapy on the patient's bacterial infection (which may be a wound infection, post-surgical infection or systemic bacteremia). Accordingly, the patient may be undergoing phage therapy for any bacterial pathogen that poses a health threat including, but not limited to the “ESKAPE” pathogens (Enterococcus faecium, Staphylococcus aureus, Klebsiella pneumonia, Acinetobacter baumannii, Pseudomonas aeruginosa and Enterobacter sp), which are often nosocomial in nature and can cause severe local and systemic infections. Among the ESKAPE pathogens, A. baumannii is a Gram-negative, capsulated, opportunistic pathogen that is easily spread in hospital intensive care units. Many A. baumannii clinical isolates are also MDR bacteria. Phage treatment of MDR bacteria (i.e. bacteria that demonstrate resistance to multiple antibacterial drugs, e.g. antibiotics) is of particular interest to the Applicant. Thus, in some preferred embodiments, the clinical samples will have been taken from patients undergoing phage therapy of an infection by MDR bacteria including, but not limited to, methicillin-resistant S. aureus (MRSA) and vancomycin-resistant Enterococci (VRE).

In another application of the invention, the method may be used with veterinary samples such as blood, serum, plasma, or urine taken from an animal subject (e.g. a livestock animal, companion animal such as a dog or cat, or exotic animal such as a lion or elephant).

The method of the invention is also suitable for use with agricultural samples such as soil, plant tissue or extracts of soil or plant tissue which may comprise, for example, phage and/or their host plant pathogenic bacteria. In this context, the method may be used to preserve samples for analysis to assess, for example, phage biocontrol treatment of crops affected with a bacterial pathogen (e.g. potato plants affected by soft rot caused by Pectobacterium or Dickeya bacterial species; tomato plants affected by bacteria wilt caused by Ralstonia solanacearum infestation; and apple and pear trees affected with fire blight (Erwinia amylovora); Buttimer C et al., Front Microbiol 8: Art. 34, 2017).

Moreover, the method of the invention is suitable for use with samples from other diverse sources including, for example, soil, water treatment plants, raw sewage, sea water, lakes, rivers, streams, standing cesspools, animal and human intestines, and fecal matter. These kinds of samples may be regarded as environmental samples. As understood herein, the term “diverse sources” includes a wide variety of different places where phage and/or bacteria may be found including, but not limited to, any place where bacteria are likely to thrive. In fact, phage are universally abundant in the environment, making the isolation of new phage very straightforward. The primary factors affecting the successful isolation of such phage are the availability of a robust collection of clinically relevant bacterial pathogens to serve as hosts, and access to diverse environmental sampling sites. The method of the invention may assist in the isolation of phage, including new phage, from environmental samples by preserving viable phage and/or bacteria (such as their host bacteria) present in the samples between collection and analysis.

The method of the invention involves rapidly lowering the temperature of the biological sample to a temperature of −50° C. or lower, preferably to a temperature of about −78° C. or lower. The temperature of dry ice (frozen carbon dioxide), which is typically readily available in a hospital or laboratory setting for instance, has a temperature of −78.5° C. Accordingly, one of skill in the art will readily appreciate that in some embodiments, the method may involve placing the biological sample into a container (e.g. a polystyrene foam cooler box) of dry ice. In the case of clinical specimens, this can be readily done at the patient's bedside immediately after the sample has been taken; thereby rapidly lowering the temperature of the sample to a temperature of about −78° C.

At a time preferably prior to the step of rapidly lowering the temperature of the sample, a suitable cryoprotectant is added. The cryoprotectant may be selected from, for example, any of the suitable cryoprotectants known to one of skill in the art including, but not limited to, glycerol, ethylene glycol, propylene glycol and dimethylsulfoxide (DMSO) and combinations thereof.

In some preferred embodiments, the cryoprotectant is glycerol. The glycerol may be added to the sample in an amount in the range of, for example, 5-50% (v/v), but preferably, in an amount in the range of 10-30% (v/v), and even more preferably, in an amount in the range of 15-25% (v/v) (e.g. 20% glycerol). In particularly preferred embodiments, the cryoprotectant is 15% glycerol, 16% glycerol, 17% glycerol, 18% glycerol, 19% glycerol, 20% glycerol, 21% glycerol, 22% glycerol, 23% glycerol, 24% glycerol or 25% glycerol.

Once the temperature of the sample has been lowered to a temperature of −50° C. or lower in the presence of a cryoprotectant, it is maintained substantially at such a temperature(s) until required for analysis. As such, during any transport (e.g. shipping) of the sample and any period(s) of storage (e.g. storage before, during and/or after transport), the sample is maintained substantially at a temperature(s) of −50° C. or lower in the presence of a cryoprotectant.

The method of the invention permits the samples to be transported and/or stored following collection such that the viability of any phage and/or bacteria present is maintained while also inhibiting potential interactions between the phage and bacteria. This allows for accurate quantitative determinations of phage and bacterial concentrations when the samples are thawed and analyzed in the laboratory (as long as they are maintained substantially at a temperature(s) of −50° C. or lower in the presence of a cryoprotectant).

Analysis of the sample may involve, for example, one or more of the standard laboratory techniques known to one of skill in the art for assaying for phage and/or bacteria.

For phage, this may simply involve preparing serial dilutions of the sample in a suitable media or solution and plating each out on a culture plate provided with a bacterial “lawn”. Following incubation of the plates under suitable conditions, phage plaques can then be scored. Similarly, for bacteria, analysis may involve, for example, preparing serial dilutions of the sample in a suitable media or solution and plating each out on a culture plate. Following incubation of the plates under suitable conditions, bacterial colonies can then be scored.

Analysis of the sample can be used for determining, for example, whether pathogenic bacteria present in the sample is sensitive to a phage therapy or phage biocontrol treatment. This may be assessed by, for example, comparing the concentration of bacteria (i.e. as may be determined by scoring of bacterial colonies as described above) in a sample taken before and after commencement of the phage therapy or biocontrol treatment—a decrease in concentration of the bacteria in the later sample would indicate that the bacteria is “responding” to the applied phage therapy or biocontrol treatment (i.e. the phage therapy or biocontrol treatment is being effective). On the other hand, if it is found that there has been no change in the bacterial concentration between the samples, or in fact an increase in the bacterial concentration in determined in the sample taken after the commencement of the phage therapy or biocontrol treatment, then the analysis may indicate that the bacteria is insensitive to the phage therapy or biocontrol treatment (i.e. the phage therapy or biocontrol treatment is ineffective). Typically, analysis will be conducted on samples taken at a number of time points after commencement of the phage therapy or biocontrol treatment. For instance, in the context of clinical specimens taken from a patient, samples for analysis would typically be taken at multiple time points (e.g. 0 hours, 1 hour, 6 hours, 12 hours and 24 hours) and the results of the analysis of all of such samples considered by the physician before making any conclusions on the effectiveness of the phage therapy on the patient's bacterial infection and/or any decision to maintain the current phage therapy or otherwise make one or more changes (including changing one or more of the phage strains and/or adding one or more additional phage strains).

The invention further relates to a kit comprising at least a container adapted to receive a biological sample, such as blood, serum, plasma, or urine, wherein said container may be pre-loaded with a suitable cryoprotectant, and wherein said kit optionally includes instructions for use of the kit in the method of invention for preserving a biological sample.

Although the invention herein has been described with reference to embodiments, it is to be understood that these embodiments, and examples provided herein, are merely illustrative of the principles and applications of the present invention. It is therefore to be understood that numerous modifications can be made to the illustrative embodiments and examples, and that other arrangements can be devised without departing from the spirit and scope of the present invention as defined by the appended claims. All patent applications, patents, literature and references cited herein are hereby incorporated by reference in their entirety.

### EXAMPLES

The invention will now be further illustrated with reference to the following examples. It will be appreciated that what follows is by way of example only and that modifications to detail may be made while still falling within the scope of the invention.

### Example 1: Bacteriophage Interaction with their Host Bacteria at Varying Temperatures

The interaction between phage and host cells in samples was investigated at varying temperatures, namely at room temperature (RT), 0° C. (ice) and −78° C. (dry ice).

Briefly, a 40 mL solution of phosphate buffered saline (PBS) containing 20% glycerol with ˜10⁴ CFU/mL of Escherichia coli bacteria (EcoIII) and ˜10⁶ PFU/mL of EcoIIIϕG phage respectively. One mL aliquots of this solution were placed into twenty-eight microtubes. These tubes were divided such that eight tubes were stored in ice, eight tubes were stored in dry ice and eight tubes were stored at RT. The remaining four tubes were used, immediately, to determine the initial concentrations of bacteria and phage present in the sample. Bacterial concentrations were determined by diluting each sample 1:10, 1:100 and 1:1000 in PBS, followed by plating on trypticase soy agar (TSA). Phage concentrations were determined by diluting each sample 1:10, 1:100, 1:1000 and 1:10000 in stabilization media (SM) buffer, followed by plating of each sample in soft agar containing an EcoIII bacterial lawn on TSA plates pre-warmed to 37° C. At one hour after the initial samples were titered, four tubes that were stored in ice (0° C.), dry ice (−78° C.) and at RT were removed for the determination of bacterial and phage concentrations. The remaining tubes were assayed for bacterial and phage concentrations at 24 hours following initiation of the experiment. All plates were incubated at 37° C. for a minimum 12 hours, before bacterial colonies and phage plaques were scored.

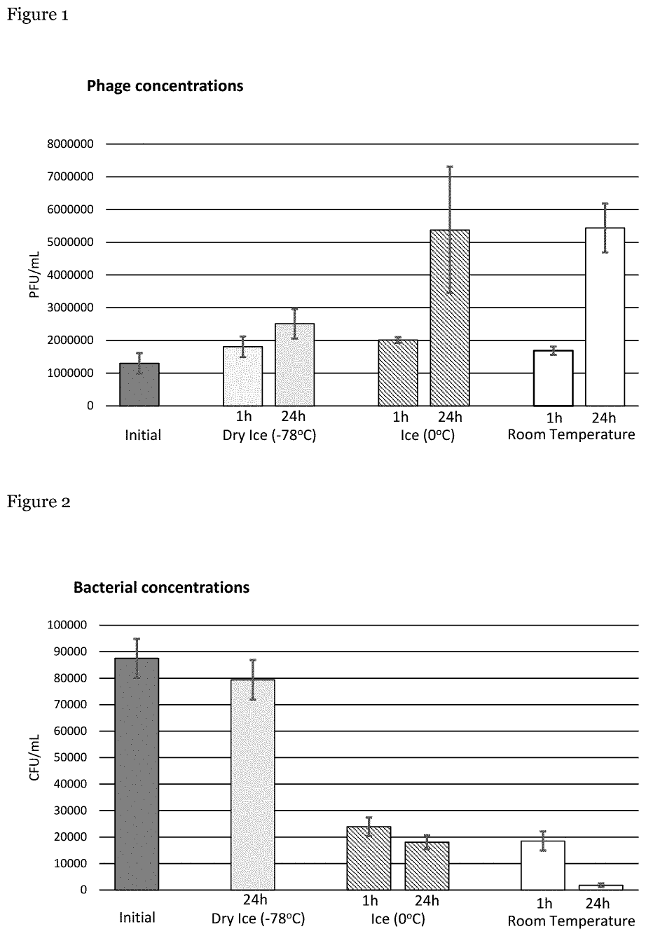

The results are shown in Tables 1 and 2 below and FIGS. 1 and 2.

Table 1 provides a summary of the phage concentrations in the presence of host bacteria at the temperatures tested in PBS with 20% glycerol.

TABLE 1
Bacteriophage interaction with their host bacteria at: room temperature,
0° C. (ice) and −78° C. (dry ice).

| | Time of | Phage Titer | |
|---|---|---|---|
| | Sampling | (PFU/mL) | Standard Deviation |
| Initial | | 1.30 × 10⁶ | ±3.14 × 10⁵ |
| −78° C. (Dry Ice) | 1 h | 1.81 × 10⁶ | ±3.16 × 10⁵ |
| | 24 h | 2.51 × 10⁶ | ±4.51 × 10⁵ |
| 0° C. (Ice) | 1 h | 2.01 × 10⁶ | ±8.54 × 10⁴ |
| | 24 h | 5.38 × 10⁶ | ±1.93 × 10⁶ |
| Room | 1 h | 1.69 × 10⁶ | ±1.24 × 10⁵ |
| Temperature | 24 h | 5.44 × 10⁶ | ±7.47 × 10⁵ |

Phage titer (PFU/mL) and Standard Deviation were calculated from quadruplicate sampling. The results shown in Table 1 and FIG. 1 show that at all temperatures, the phage titer increased over 24 hours, however the increase was considerably less under storage at −78° C. (dry ice) in PBS with 20% glycerol. Thus, keeping samples at RT or in 0° C. (ice) is insufficient to prevent phage replication and indicates that clinical specimens would need to be stored at −78° C. (dry ice) or lower.

TABLE 2
Bacteriophage interaction with their host bacteria at room
temperature, 0° C. (ice) and −78° C. (dry ice).

| | | Bacterial conc. | Standard |
|---|---|---|---|
| | Time of Sampling | (CFU/mL) | Deviation |
| Initial | | 8.75 × 10⁴ | ±7.36 × 10³ |
| −78° C. (Dry Ice) | 1 hour | 2.31 × 10⁴ | ±2.93 × 10³ |
| | 24 hours | 7.94 × 10⁴ | ±7.47 × 10³ |
| 0° C. (Ice) | 1 hour | 2.39 × 10⁴ | ±3.52 × 10³ |
| | 24 hours | 1.81 × 10⁴ | ±2.61 × 10³ |
| Room | 1 hour | 1.85 × 10⁴ | ±3.62 × 10³ |
| Temperature | 24 hours | 1.81 × 10³ | ±6.88 × 10² |

Table 2 provides a summary of the bacterial concentrations in the presence of phage at the temperatures tested in PBS with 20% glycerol. Bacterial concentrations (CFU/mL) and standard deviations were calculated from quadruplicate sampling. The results are shown graphically in FIG. 2. This figure illustrates the need to use a temperature of −78° C. (dry ice) or lower to prevent phage replication which results in bacterial destruction in clinical specimens.

### Example 2: Preservation of Samples from Patients Treated with Therapeutic Bacteriophage

Experimentation was conducted to identify and develop a method to preserve clinical samples (particularly, urine specimens) from patients treated with therapeutic phage. Following the results obtained in Example 1, the method involved using low temperature (−78° C. (dry ice)) to inhibit the interaction of the phage with the patient's host bacteria present in samples until they can be quantitatively examined in a laboratory.

Urine was provided by male volunteers who were free of antibiotics. These volunteers were instructed to follow clean catch urine specimen collection procedures. To preserve bacterial cell viability and phage titers, glycerol was immediately added to each urine sample to provide a 20% v/v final concentration of glycerol. Urine specimens were examined using three different pHs.

E. coli (EcoIII) bacteria and EcoIIIϕG phage were added to each urine sample to achieve a final concentration of bacteria and phage of 10⁵ CFU/mL and 10⁵ PFU/mL respectively. All of the samples were thoroughly mixed, and aliquots were removed for immediate assaying in quintuplicate to verify initial phage titers and bacterial concentrations. The remaining sample aliquots were then rapidly frozen on dry ice. After initial freezing, urine samples were assayed at 1 hour and 24 hours as follows: five samples, at each time point, were removed from the dry ice and diluted 1:10, 1:100, and 1:1000 in cold PBS. From each of the three serial dilutions, a 100 μL aliquot was spread plated on TSA plates for the development of bacterial colonies to determine the bacterial concentrations. Determinations of phage concentrations were measured by performing serial dilutions in SM buffer followed by addition and mixing of the dilutions with soft agar containing an EcoIII bacterial lawn. This soft agar was then evenly poured onto pre-warmed (37° C.) TSA plates for development of phage plaques. All samples were incubated at 37° C. for a minimum 12 hours, after which the bacterial colonies and phage plaques were scored.

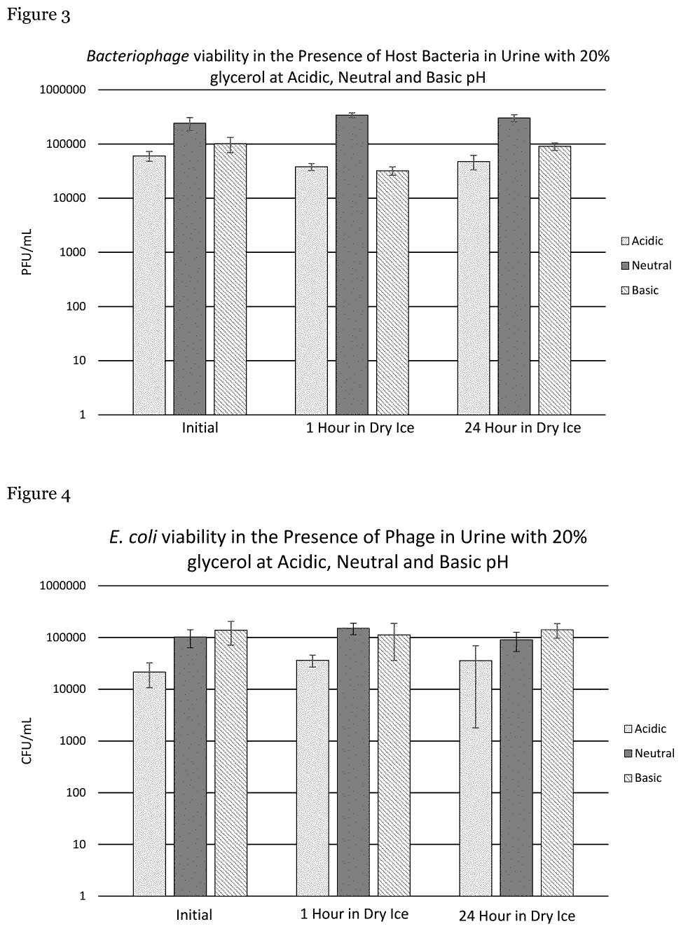

The results are shown in Tables 3 and 4 below and FIGS. 3 and 4.

Table 3 provides a summary of the phage titers (in the presence of bacteria) in urine specimens determined from: the initial sample, 1 hour in dry ice and after 24 hours in dry ice. The measurements were made in acidic, neutral and basic pH urine samples.

TABLE 3
Summary of Bacteriophage Titer in the Presence of Host Bacteria
in urine with 20% glycerol at Acidic, Neutral and Basic pH

| | | Initial Concentrations | | 1 Hour in Dry Ice | | 24 Hours in Dry Ice | |
|---|---|---|---|---|---|---|---|
| | | | Standard | | Standard | | Standard |
| pH | | PFU/mL | Deviations | PFU/mL | Deviations | PFU/mL | Deviations |
| 5.4 | (Acidic) | 6.02 × 10⁴ | ±1.22 × 10⁴ | 3.80 × 10⁴ | ±5.65 × 10³ | 4.74 × 10⁴ | ±1.41 × 10⁴ |
| 6.98 | (Neutral) | 2.42 × 10⁵ | ±6.52 × 10⁴ | 3.41 × 10⁵ | ±3.35 × 10⁴ | 3.02 × 10⁵ | ±4.38 × 10⁴ |
| 8.15 | (Basic) | 1.38 × 10⁵ | ±6.69 × 10⁴ | 1.12 × 10⁵ | ±7.57 × 10⁴ | 1.41 × 10⁵ | ±4.40 × 10⁴ |

These results are also shown graphically in FIG. 3. The figure illustrates that at each pH tested, there was good preservation of viable phage, particularly after 24 hours storage, where phage concentration recovered to a number substantially equivalent to the initial phage concentration (especially with the acidic and basic samples, which after 1 hour storage showed a decrease in phage concentration).

Table 4 provides a summary of the bacterial concentrations (in the presence of phage) in urine specimens determined from: the initial sample, 1 hour in dry ice and after 24 hours in dry ice. The measurements were made in acidic, neutral and basic pH urine samples. Bacterial concentrations (CFU/ml) were determined from quintuplicate sampling.

TABLE 4
Summary of bacterial cell viability in the presence of bacteriophage
in urine with 20% glycerol at acidic, neutral and basic pH

| | | Initial Concentrations | | 1 Hour in Dry Ice | | 24 Hours in Dry Ice | |
|---|---|---|---|---|---|---|---|
| | | | Standard | | Standard | | Standard |
| pH | | CFU/mL | Deviations | CFU/mL | Deviations | CFU/mL | Deviations |
| 5.4 | (Acidic) | 2.15 × 10⁴ | ±1.08 × 10⁴ | 3.62 × 10⁴ | ±9.42 × 10³ | 3.57 × 10⁴ | ±3.39 × 10⁴ |
| 6.98 | (Neutral) | 1.02 × 10⁵ | ±3.88 × 10⁴ | 1.51 × 10⁵ | ±3.81 × 10⁴ | 9.00 × 10⁴ | ±3.63 × 10⁴ |
| 8.15 | (Basic) | 1.38 × 10⁵ | ±6.69 × 10⁴ | 1.12 × 10⁵ | ±7.57 × 10⁴ | 1.41 × 10⁵ | ±4.40 × 10⁴ |

FIG. 4 shows these results graphically. The figure illustrates that at each pH tested, there was good preservation of viable E. coli bacteria after 1 hour and 24 hours storage in PBS with 20% glycerol at −78° C. (dry ice).

### CONCLUSIONS

The experiments described in Examples 1 and 2, using PBS containing 20% glycerol, examined the stability of mixtures of phage and bacterial host concentrations at: −78° C. (dry ice), 0° C. (ice) and RT. Samples stored at 0° C. and at RT proved to be unstable with time, as observed by the increased phage titers and the decreased bacterial titers, relative to the initial sample concentrations (see FIGS. 1 and 2). These results demonstrated that phage can infect and replicate in its host bacteria when samples are stored on ice (0° C.) or RT, but in contrast, phage titer and bacterial concentrations were maintained at their initial values when samples were stored −78° C. (dry ice). Further, it was found that these results were not substantially affected by the pH of the sample.

Accordingly, the present invention offers a novel and simple method of preserving the concentrations of mixtures of phage and their host bacteria in clinical specimens (especially urine) that may be employed immediately after collection (e.g. at the bedside) for later analysis.

The invention is not limited to the embodiment herein before described which may be varied in construction and detail without departing from the spirit of the invention. The entire teachings of any patents, patent applications or other publications referred to herein are incorporated by reference herein as if fully set forth herein.

1. A method of preserving a biological sample comprising, or suspected of comprising, one or more bacteriophage (phage), the method comprising rapidly lowering the temperature of the sample (e.g. freezing) to a temperature of −50° C. or lower in the presence of a suitable cryoprotectant.

2. The method of claim 1, wherein biological sample is selected from clinical specimens.

3. The method of claim 1, wherein the biological sample is selected from veterinary samples, agricultural samples and environmental samples.

4. The method of claim 1, wherein the biological sample comprises a mixture of one or more phage and their host bacteria.

5. The method of claim 1, wherein the biological sample is blood, plasma, serum or urine.

6. The method of claim 1, wherein the temperature of the biological sample is lowered to a temperature of about −78° C. or lower.

7. The method of claim 6, wherein the temperature of the biological sample is lowered to a temperature of about −78° C. by placing the sample in dry ice.

8. The method of claim 1, wherein the cryoprotectant is selected from the group consisting of glycerol, ethylene glycol, propylene glycol and dimethylsulfoxide (DMSO) and combinations thereof.

9. The method of claim 8, wherein the cryoprotectant is glycerol.

10. The method of claim 9, wherein the glycerol is added to the sample in an amount in the range of 10-30% (v/v).

11. The method of claim 9, wherein the glycerol is added to the sample in an amount in the range of 15-25% (v/v).

12. The method of claim 8, wherein the cryoprotectant is 20% glycerol.

13. The method of claim 1, wherein the biological sample is a clinical sample taken from a patient undergoing phage therapy for a bacterial infection.

14. The method of claim 13, wherein the patient is undergoing phage therapy for an infection by one or more of the “ESKAPE” pathogens.

15. The method of claim 13, wherein the patient is undergoing phage therapy for an infection by one or more multiple drug resistant (MDR) bacteria.

16. The method of claim 13, wherein the clinical sample is urine.

17. The method of claim 16, wherein the temperature of the urine sample is lowered to a temperature of about −78° C. by placing the sample in dry ice.

18. The method of claim 17, wherein the cryoprotectant is glycerol and is added to the sample in an amount in the range of 10-30% (v/v).

19. The method of claim 17, wherein the cryoprotectant is 20% glycerol.

20. A kit comprising at least a container adapted to receive a biological sample, wherein said container is pre-loaded with a suitable cryoprotectant, and wherein said kit optionally includes instructions for use of the kit in the method of claim 1.
